(19) United States
(12) Patent Application Publication
(10) Pub. No.: US 2024/0081736 A1
(43) Pub. Date: Mar. 14, 2024

(54) IN-EAR NOISE, IMPACT, AND BLAST EXPOSURE MONITOR

(71) Applicant: PAXAURIS LLC, Phoenix, AZ (US)

(72) Inventors: Anthony DIETZ, Phoenix, AZ (US); Sean WELLS-RUTHERFORD, Phoenix, AZ (US)

(21) Appl. No.: 18/244,867

(22) Filed: Sep. 11, 2023

Related U.S. Application Data
(60) Provisional application No. 63/405,361, filed on Sep. 9, 2022.

(51) Int. Cl. A61B 5/00; A61F 11/08; H04R 1/10

(52) U.S. Cl. CPC A61B 5/6817; A61F 11/085; H04R 1/1016

(57) ABSTRACT

A hearable system can comprise eartips for hearing protection, instrumented earpieces for measuring exposure to continuous and impulse noise, head impact, and blast while also providing hear-through and wireless communication capabilities, and a monitor for recording exposure data, transmitting communications, and warning of exceedances. Introducing motion sensors for impact detection to an in-ear noise dosimeter enables methods for rejecting false noise exposure events. The pressure sensitivity of the motion sensors used for impact detection also enables blast measurement, with the different frequency characteristics of impact and blast events being used to distinguish between the two when calculating exposure metrics. Deriving both pressure and motion from a single sensor offers an attractive solution for an in-ear earplug, earpiece, or hearable capable of measuring noise, impact, and blast exposure.

### CROSS-REFERENCE TO RELATED APPLICATIONS

The present Application claims priority to and the benefit of U.S. Provisional Patent Application No. 63/405,361, filed Sep. 9, 2022, which is fully incorporated herein by reference.

### FIELD

The present invention relates generally to methods and devices for detecting and monitoring exposure to conditions that harm hearing and brain health such as head impacts, noise, impulse sound, and blast waves. In particular, the present invention relates to an in-ear device or system that provides hearing protection with hear-through transparency while also monitoring noise, impact, and blast exposure. The invention employs the pressure sensitivity of the motion sensors used for impact detection to also detect blast events and separates impact events from blast events through the application of frequency-dependent filters. The addition of motion sensors for impact detection to the in-ear device also enables methods to reject false noise exposure events. Redundancy between the left and right sensor suites is further used to detect and eliminate false noise, impact, and blast exposure events.

### BACKGROUND

Wearable technology can increase worker safety by monitoring environmental and physiological parameters to identify potentially harmful situations that put workers' health at risk. The most effective wearables are those that both monitor and protect, justifying the encumbrance of the monitor by the protection it provides.

Two critical injuries suffered by military service members and industrial workers are hearing damage and traumatic brain injury (TBI). Hearing damage can be caused by exposure to continuous noise, impulse noise, and blasts. TBI can be caused by head impacts and blasts.

Head and hearing protection in the form of earplugs, earmuffs, and helmets are worn to reduce the risk of hearing damage and TBI. An individual's risk of injury depends on the magnitude and duration of exposure and the degree of protection provided. In each case, cumulative exposure exacerbates the risk.

According to recent information, some 22 million U.S. workers are at risk of noise-induced hearing loss, and over $240 M is paid out each year for hearing-related workers compensation claims. However, the workplace noise surveys required by OSHA only identify when hearing protection should be worn. They do not evaluate whether workers are wearing their hearing protection properly or the level of protection being provided to each individual, which depends on the hearing protection device being used and whether it is being used properly. Even body-worn noise dosimeters fail to provide this information as they only measure the ambient noise without any information regarding the use of hearing protection. Only an in-ear dosimeter can provide the critical data needed to evaluate hearing protection performance in the field and to improve training and compliance.

TBIs resulting from head impacts are one of the most common and costly workplace injuries, accounting for 22% of all work-related injury fatalities between 2003 and 2008, and 46% of work-related fatal falls, according to recent information. Furthermore, concussions and TBIs are underreported, making it hard to target prevention programs. Improved surveillance programs are needed with accurate injury identification and TBI risk metrics. Helmet-mounted instrumentation is inadequate as it can suffer inaccuracies due to head-relative helmet motion. Even head-mounted instrumentation such as head bands and skin patches can exhibit poor coupling due to scalp motion. Instrumented earplugs offer an attractive alternative. The ear canal penetrates the skull, and a secure earplug is well coupled to the head and close to the injury site in the brain.

Integrating microphones, pressure sensors, and motion sensors into an earplug or earpiece, would result in a hearable device that both provides protection and a monitoring capability for noise, head impact, and blast exposure. However, for the hearable to provide effective protection and monitoring, it must be fitted correctly and worn consistently, which means it must be easy to fit and comfortable, and it must not extend excessively beyond the ear. Wireless operation is also important, to reduce snag hazards, interference with other personal protective equipment, and annoyance. As a result, size and weight are important design constraints that limit sensor choice, processor power, and battery size.

One challenge is the range of pressures and motion that must be measured to adequately capture the exposure risks. Continuous noise exposure presents a risk of hearing damage at levels above 85 dBA, while survivable blast overpressures may exceed 210 dBA. As dB is a logarithmic scale, this corresponds to a very large pressure range, from 0.36 Pa (0.00005 psi) to 700 kPa (100 psi), much larger than any single sensor and data acquisition channel can capture. A microphone would need to be coupled with a pressure transducer to capture the full range. Head motion includes both linear and angular motion in three axes, requiring a six degrees of freedom motion system such as three linear accelerometers and three gyroscopes to capture the full range of head motion. Furthermore, these sensors need adequate bandwidth to capture the needed risk metrics, and each requires space and power.

Another challenge is eliminating false detections. Knocking or bumping the earpiece may produce a microphone signal similar to that from an impulse noise or a blast event. One solution for continuous or impulse noise dosimeters is to use accelerometers to detect such artifacts and only include events without a correlated accelerometer event. However, accelerometers have varying degrees of pressure sensitivity, and high-pressure impulse and blast events will produce an accelerometer response that might cause valid events to be rejected.

Hearing protection for military or industrial personnel should also include a level-dependent hear-through capability or transparency mode to provide environment sounds at safe levels for situational awareness. In-ear communications are also often desired for increased intelligibility. The transducers and processing required for these capabilities put additional burdens on the available space and power in an earpiece.

Space and power constraints limit the number of sensors that can be included in a hearable. As a result, there is a need for limited sensor suites able to capture the full range of pressure and head motion.

### SUMMARY

While in-ear devices for measuring noise and impulse exposure have been developed, and devices for measuring head impacts in the ear have been described, none have combined both noise and impact measurement capabilities. The present invention comprises such a combination, which enables a new method for measuring exposure to blast overpressures without the need for an additional pressure sensor as well as a method to distinguish impact and blast events from each other. The reduced sensor suite enables the integration of hear-through transparency and wireless data and voice communications in a compact, comfortable earpiece. Furthermore, the addition of motion sensors for impact detection enables methods for eliminating false impulse and blast events, while the integration of noise and hear-through sensors enables a fit check to ensure the earpiece is secure, which is necessary for accurate impact measurement.

Integrating microphones and high-bandwidth motion sensors into wireless hearable earpieces enables both in-ear dosimetry for measuring exposure to continuous and impulse noise as well as exposure to head impacts and blast overpressures. In particular, deriving both pressure and motion from a single sensor offers an attractive solution for an in-ear earplug, earpiece, or hearable capable of measuring noise, impact, and blast exposure. Embodiments of the present invention include a hearable system comprising eartips for hearing protection, instrumented earpieces for measuring exposure to noise, head impact, and blast while also providing hear-through and wireless communication capabilities, and a monitor for recording exposure data, transmitting communications, and warning of exceedances.

An object of the invention is to determine when the earplug is being worn so that exposure is appropriately categorized between protected (hearable is properly fitted) and unprotected (hearable is not properly fitted) conditions. Head impact events are only characterized and included in the risk metrics when the earplug is properly fitted and providing protection as this is the only time it is well coupled to the head. The hearable is correctly fitted when an acoustic seal is established by the eartip, which will result in a difference in the sound level between the internal dosimetry microphone and the external hear-through microphone. Hence, comparing the sound level of these two microphones will give an indication of fit. A fit threshold may be chosen that depends on the attenuation rating of the eartip. Noise, impact, and blast events are only included in the risk metrics when this threshold is exceeded.

Another object of the invention is to determine between true impact or blast events and false detections by comparing the levels detected at each ear. Both ears must detect an event and the level of each should be within predetermined levels for the event to be accepted as true. The level thresholds depend on head shadowing and transcranial transmission and depend on whether the detected event is an impact or blast event.

The above summary is not intended to describe each illustrated embodiment, claimed embodiment or implementation of the invention. The detailed technology and preferred embodiments implemented for the subject invention are described in the following paragraphs accompanying the appended drawings for people skilled in this field to well appreciate the features of the claimed invention. It is understood that the features mentioned hereinbefore and those to be commented on hereinafter may be used not only in the specified combinations, but also in other combinations or in isolation, without departing from the spirit and scope of the present invention.

### BRIEF DESCRIPTION OF THE DRAWINGS

The invention may be more completely understood in consideration of the following detailed description of various embodiments of the invention in connection with the accompanying drawings, in which:

FIG. 1 shows a section of an ear with a hearable earpiece fitting comfortably into the concha region and the earpiece sound port extending into the ear canal with the eartip sealing the ear canal, in accordance with embodiments of the present invention.

FIG. 2A shows a side view of a hearable earpiece housing with acoustic port, in accordance with embodiments of the present invention.

FIG. 2B shows a side view of a hearable earpiece housing with foam eartip mounted on acoustic port, in accordance with embodiments of the present invention.

FIG. 2C shows a side view of components and internal rigid-flex circuits of a hearable earpiece, in accordance with embodiments of the present invention.

FIG. 3 shows a block diagram of a hearable circuit, in accordance with embodiments of the present invention.

FIG. 4 shows inputs and outputs of noise exposure functions including those for dosimetry and impulse detection and measurement, in accordance with embodiments of the present invention.

FIG. 5 shows inputs and outputs of a hear-through function, in accordance with embodiments of the present invention.

FIG. 6 shows a flow chart of impact and blast detection and measurement functions, in accordance with embodiments of the present invention.

FIG. 7 shows a response of an earplug accelerometer fitted into the ear of a biofidelic head subject to vibration forcing on its forehead from a shaker excited by broad band white noise, in accordance with embodiments of the present invention.

FIG. 8 shows the response of an earplug accelerometer fitted into the ear of a biofidelic head subject to acoustic forcing from an external loudspeaker driven by broad band white noise, in accordance with embodiments of the present invention.

FIG. 9 shows the response of a biofidelic head to impulse sound from a starter's pistol, in accordance with embodiments of the present invention. The incident pressure signal from an external microphone is plotted, as well as the acceleration recorded by an earplug accelerometer fitted into the ear of the head and the pressure from a hydrophone positioned in the brain of the head.

FIG. 10 shows the correlation between an earplug accelerometer fitted into the ear of a biofidelic head and a hydrophone positioned in the brain of the head, when the head is subject to impulse sounds of three different levels from a starter's pistol, in accordance with embodiments of the present invention. A high degree of correlation is evident in the plot.

FIG. 11A shows the external pressure signal from a stater's pistol recorded by a microphone positioned adjacent to the ear of a biofidelic head, in accordance with embodiments of the present invention.

FIG. 11B shows signals from the three axes of a triaxial accelerometer mounted in the earpiece of a hearable fitted into the ear of a biofidelic head exposed to the impulse sound from a starter's pistol that produced the pressure signal shown in FIG. 11A, in accordance with embodiments of the present invention.

FIG. 11C shows signals from the three axes of a triaxial gyroscope mounted in the earpiece of a hearable fitted into the ear of a biofidelic head exposed to the impulse sound from a starter's pistol that produced the pressure signal shown in FIG. 11A, in accordance with embodiments of the present invention.

FIG. 12 shows a correlation between the peak acceleration magnitude recorded by a triaxial accelerometer mounted in an earpiece and fitted into a model ear and the peak overpressure measured by a reference transducer adjacent to the ear, in accordance with embodiments of the present invention. Low pressures were generated by a starter's pistol and high pressures were generated by a shock tube.

FIG. 13 shows the hearable system including hearable earpieces with foam eartips fitted, a head-mounted monitor, and a charging case, in accordance with embodiments of the present invention. The hearables transmit exposure data to the monitor over a near field communication (NFC) or like connection. The monitor then transmits risk metrics over Bluetooth or like connections to a base station.

FIG. 14 shows a flow chart of the monitor processing functions including checks to identify false noise, impact, or blast events, in accordance with embodiments of the present invention.

FIG. 15 shows a flow chart of the monitor processing functions for rejecting false detections and determining when the earpieces are being worn and correctly fitted.

FIG. 16 shows a flow chart of the hearable system showing the data and logic flow between the components and functions.

While the invention is amenable to various modifications and alternative forms, specifics thereof have been shown by way of example in the drawings, are not intended to be to scale, and will be described in detail. It should be understood, however, that the intention is not to limit the invention to the particular embodiments described. On the contrary, the invention is to cover all modifications, equivalents, and alternatives falling within the spirit and scope of the invention as defined by the appended claims.

### DETAILED DESCRIPTION

The hearable system and device of the present invention enables wireless monitoring of exposure to noise, head impact, and blast while also providing hearing protection with hear-through transparency and in-ear communications.

The number of sensors within the present invention can be reduced to meet space and power constraints by eliminating the need for a blast pressure sensor by extracting blast overpressure measurements from the earpiece motion sensors, which also exhibit a pressure sensitivity, and using knowledge of head dynamics to separate motion from pressure in frequency space.

The earpiece housing is shaped to fit comfortably in the concha region of the wearer's outer ear. The housing includes a sound port, which is a hollow barbed tube that projects from the lower part of the housing and extends into the wearer's ear canal.

The eartip may be foam, flanged, custom, inflatable, or other type of tip. The eartip is mounted onto the sound port. When fitting the hearable, the eartip is inserted into the wearer's ear canal, and positioned so that it forms an acoustic seal. The hollow sound port extends through the ear tip so that it is in communication with the inner portion of the ear canal beyond the eartip.

Noise exposure is measured by an internal microphone mounted in the base of the earpiece and connected to the sound port to record the sound level in the wearer's ear canal inside the hearing protection provided by the eartip. The microphone is connected to an analog-to-digital converter and the signal is converted to sound level and noise dose parameters by algorithms running in a digital signal processor (DSP). Risk metrics are transmitted wirelessly to the monitor via a transceiver and antenna, with data flow controlled by a microcontroller (MCU). Power is provided by a rechargeable battery and regulated by a power management circuit. The internal microphone can measure sound levels up to approximately 150 dB SPL.

The internal microphone is positioned in the lower housing as close to the sound port as possible to minimize undesirable acoustic standing waves and resonances occurring in the ear canal and the tube or channel connecting the sound port to the microphone. The frequencies of these resonances are related to the length of the ear canal and the tube, with longer lengths causing resonances at lower frequencies. Ideally in various embodiments, sound exposure would be measured at the end of the ear canal at the tympanic membrane, which is the first part of the body's sound transduction mechanism. For this reason, the sound port extends as deep into the ear canal as comfortable, and the internal microphone is positioned as close to the sound port as possible, to minimize distance from the tympanic membrane to the internal microphone.

Hear-through is provided by an external microphone mounted over a hole in the earpiece housing positioned outside the ear canal so that it senses exterior environmental sounds. The external microphone is connected via an analog-to-digital converter to the DSP, which in turn drives a speaker connected to the sound port to deliver environmental sounds into the wearer's ear canal inside the hearing protection provided by the eartip. A dynamic range compression algorithm runs on the DSP to limit the environmental sound levels to safe levels, such as 85 dBA. As with the internal microphone, the speaker is mounted as close to the sound port as possible to minimize acoustic resonances that would interfere with the quality of sound delivered by the speaker.

Two-way, in-ear communications are provided by the speaker and by a bone-conduction microphone mounted in the housing to pick up the wearer's voice with communications signals being transmitted over the wireless link.

Head impacts are detected by motion sensors measuring linear acceleration and angular rate. Linear acceleration is measured by a high-range triaxial accelerometer chip. Angular motion is measured by an Inertial Measurement Unit (IMU) comprising a three-axis gyroscope and a low-range triaxial accelerometer. Both are digital sensor chips connected to the DSP via a digital bus. Algorithms on the DSP determine head impact risk metrics such as peak linear acceleration and peak angular rate, while rejecting false peaks that do not correspond to impacts.

Blast pressures may be too large to be detected by the internal microphone, which has an acoustic overload pressure of approximately 150 dB SPL (0.36 Pa, 0.00005 psi). A pressure transducer may be added to measure the blast overpressures, with a range up to 210 dBA (700 kPa, 100 psi). Like the internal microphone and the speaker, this pressure transducer would be connected to the sound port to measure the overpressure in the ear canal. However, the space near the sound port is limited and additional tubing to create the connection would introduce further undesirable resonances. Furthermore, miniature pressure transducers are either too large for this space or too costly for a hearable, and they introduce additional data acquisition, processing, and power burdens. To introduce a pressure transducer, some other capability would have to be dropped.

The accelerometer and the gyroscope also exhibit a pressure sensitivity, and their pressure response is measurable at the high overpressures associated with blast waves. The sensor's pressure response may be distinguished from their head motion response by considering human head dynamics. The inertia of the head means the motion associated with survivable head impacts occurs in a frequency range below 100 Hz. The head's response to blast waves is broad band with modes at frequencies well above 100 Hz. Low-pass filtering the motion sensor signals and comparing the result to the broadband signal enables the separation and categorization of impact events from blast events.

Exposure data are received and recorded by the monitor. The monitor can provide visual and tactile warnings to the wearer if exposure thresholds are exceeded. The monitor can also transmit exposure warnings to the hearables for aural communication to the wearer. The monitor can transmit and receive synchronization signals to and from the hearables and the monitor uses these to correlate events received from each hearable.

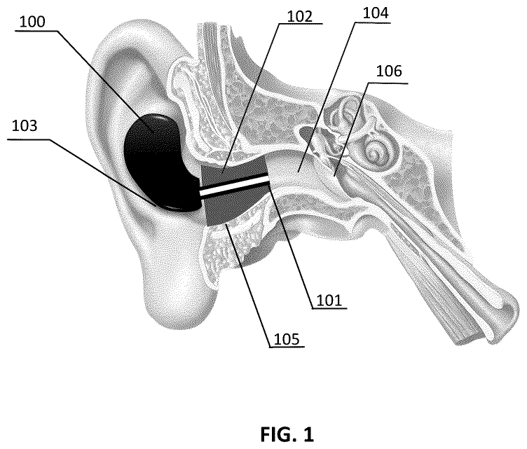

Referring to FIG. 1, in various preferred embodiments, the hearable device comprises an earpiece 100 with a sound port 101 projecting from the housing, and an eartip 102 mounted onto the sound port 101. The earpiece 100 is shaped to fit comfortably in the concha 103 of the outer ear while the sound port 101 is positioned so that it extends into the ear canal 104 so that the eartip 102 forms an acoustic seal with the walls of the ear canal 105. The hollow tube through the sound port opens into the ear canal 105 close to the tympanic membrane 106, which terminates the ear canal 104 and is the first part of the ear's sound transduction mechanism.

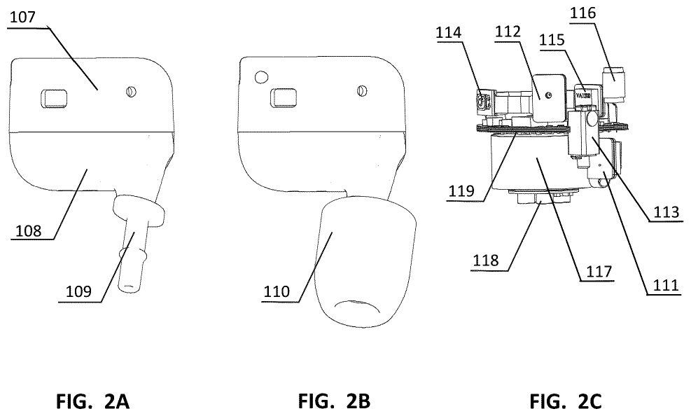

In one embodiment, as shown in FIGS. 2A-2C, the housing comprises an upper section 107 and a lower section 108, with a barbed or threaded sound port 109 projecting from the lower section 108. In this embodiment, the eartip 110 comprises a foam cylinder with a hollow elastomer tube at its center. The eartip 110 is mounted onto the hearable by inserting the sound port 109 into the hollow tube at the center of the eartip. The barbs or threads on the sound port 109 form an acoustic seal inside the eartip's elastomer tube. When the hearable is worn, the foam eartip forms a seal in the wearer's ear canal, thus also sealing the sound port to the interior of the ear canal.

In other embodiments, the eartip 110 may be a flanged type, where double or triple cylindrical elastomer flanges form the seal in the ear canal, or it may be a custom-fitted type where a solid eartip is shaped to seal an individual's ear canal. In another embodiment, a flexible silicone eartip is filled with fluid and inflated to form the acoustic seal.

The hearable components, as shown in FIG. 2C, are arranged to fit within the hearable housing. Primary components include an internal microphone 111 for noise dosimetry, an external microphone 112 and internal speaker 113 for hear-through, a button 114 for control, a bone conduction microphone 115 for communication voice pickup, a wireless antenna 116, a battery 117, and charging contacts 118. Additional components including a triaxial accelerometer, IMU chip containing a triaxial gyroscope, DSP, microcontroller, and power management circuit are mounted on the circuit board 119.

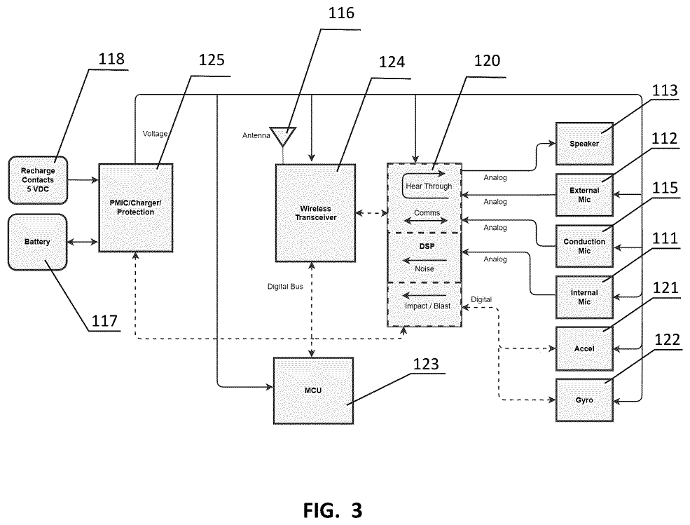

A block diagram showing the hearable earpiece architecture and connection of the components is shown in FIG. 3. The internal microphone 111, the external microphone 112, and the bone conduction microphone 115 are operatively connected to analog inputs on the DSP 120. The speaker 113 is operatively connected to an analog output on the DSP 120. The accelerometer 121 and the gyroscope 122 are operatively connected to digital inputs on the DSP 120. The DSP 120 is operatively connected via a digital bus to the microcontroller unit 123 and the wireless transceiver 124, which is operatively connected to the antenna 116. The battery 117 provides power via the power management circuit 125 and may be recharged via the recharging contacts 118.

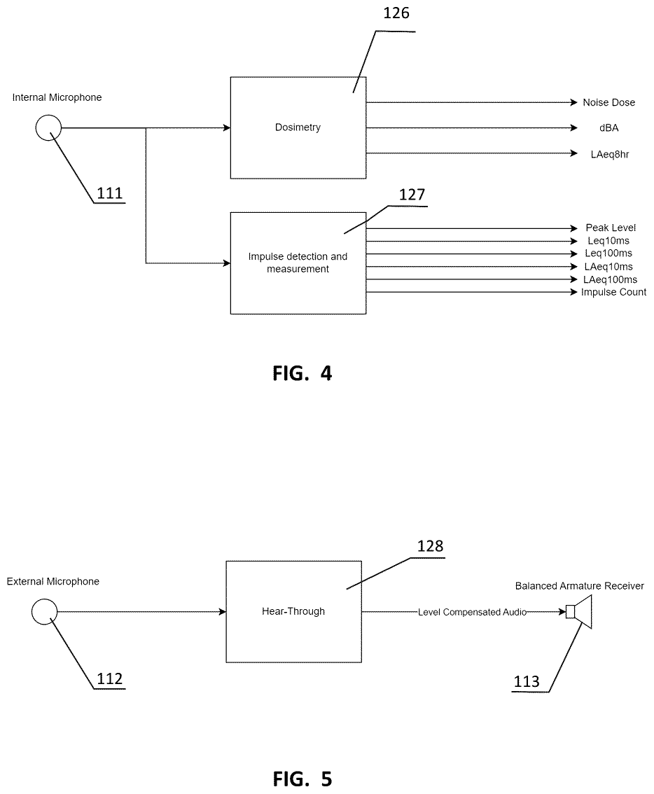

Various specific algorithms and functions are used to process the sensor signals into exposure risk metrics. The noise exposure dosimetry function 126, shown in FIG. 4, processes the signal from the internal microphone 111 and calculates specific metrics for continuous noise, such as noise dose, A-weighted sound pressure level, and equivalent sound pressure level. This algorithm is an implementation of the noise dosimetry method specified by ANSI S1.25-1991, Specification for Personal Noise Dosimeters. The impulse function 127, included in FIG. 4, also processes the signal from the internal microphone 111. This function detects acoustic impulse events, such as those from weapons firing, and computes risk metrics such as number of impulses and peak sound pressure level, among others.

The hear-through function 128, as shown in FIG. 5, processes the signal from the external microphone 112 to produce the level-dependent signal output from the speaker 113. The gain applied to the signal from the external microphone is progressively reduced to keep the signal presented to the wearer through the speaker under a safe threshold. In an exemplary embodiment, this threshold is set to 85 dBA, which is the level recommended by the National Institute for Occupational Safety and Health (NIOSH) for eight hours of exposure.

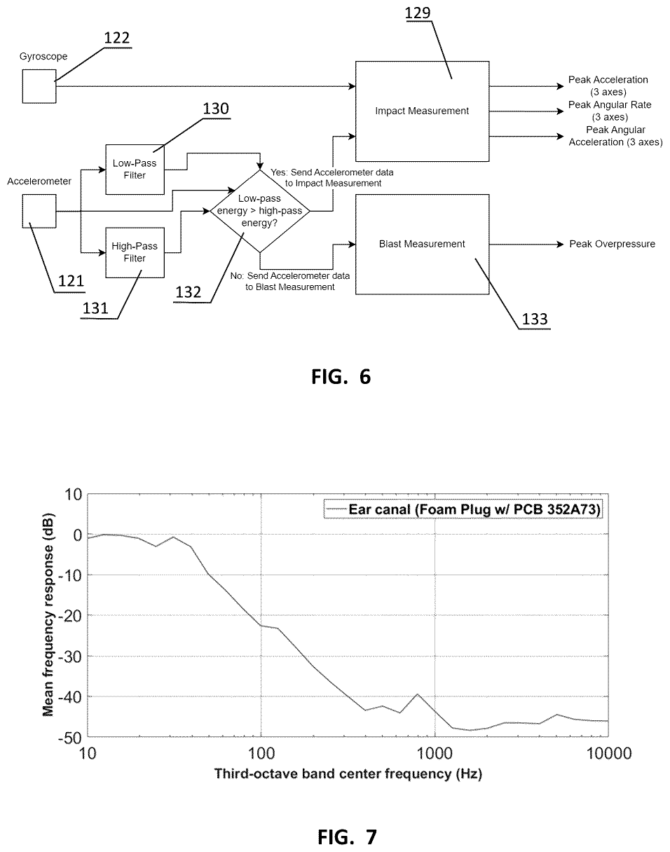

The impact function 129, as shown in FIG. 6, processes the signals from the tri-axial gyroscope 122 and the signals from the tri-axial accelerometer 121 to calculate impact risk metrics, such as peak linear acceleration and peak angular rate. Because the sensors respond to both impact and blast events, an embodiment of the invention includes an algorithm that uses an analysis of the frequency content of said events to distinguish between impact and blast events. In the embodiment depicted in FIG. 6, the accelerometer signal is split and processed independently through a low-pass filter 130 and a high-pass filter 131. Events with greater low frequency energy are classified as impacts and those with greater high frequency energy are classified as blast events 132. The blast events are processed in the blast algorithm 133 to calculate blast risk metrics such as peak overpressure in the brain. This overpressure is not directly measured but is the result of a correlation between the accelerometer response and the head and brain response. This correlation is determined from tests of instrumented heads fitted with hearables.

In one embodiment, the cutoff frequency selected is 100 Hz. This frequency was determined from tests with a biofidelic head simulator. This simulator represents the highest level of anatomic fidelity outside of testing with human test subjects or cadavers. It includes bone, brain, and skin structure segmented from a human CT scan and then simulated by a three-dimensional printed skull with the cavity filled with silicone gel and silicon skin tissue cast over the bone. The simulant's material properties are selected to match those of human bone, brain, and skin so that the head responds to acoustic and impact forcing in a similar manner to a human head. Unlike a living human head, the simulator can be instrumented with microphones at the location of the tympanic membrane and a hydrophone in the skull cavity.

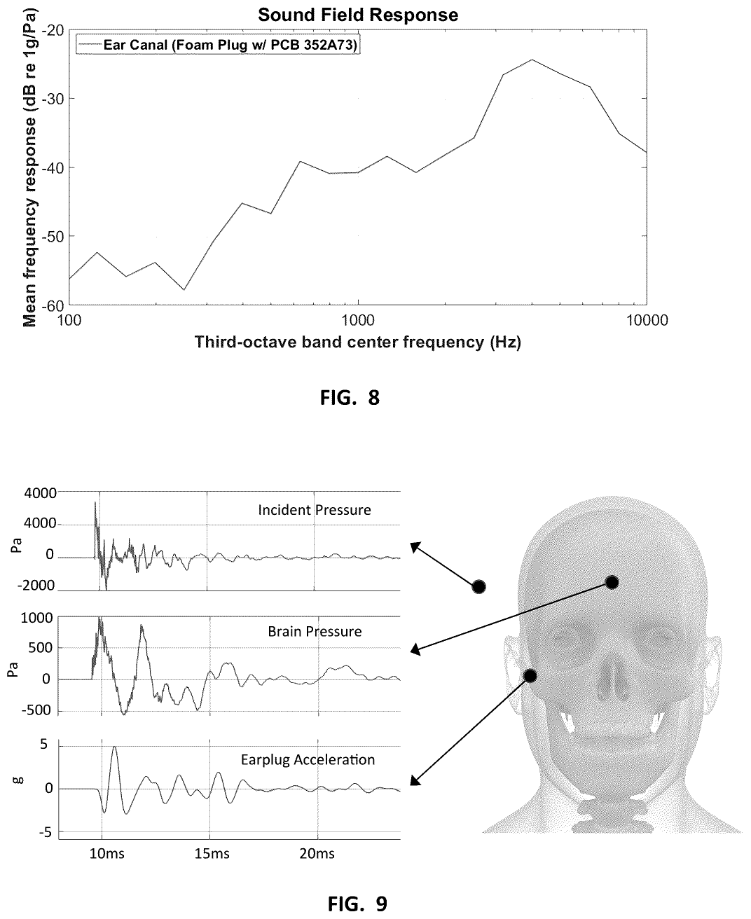

The impact frequency response of a single-axis, high-bandwidth accelerometer mounted in a foam earplug and inserted into the ear canal of the head simulator was measured by driving the head with a shaker excited by broadband white noise. The result, as shown in FIG. 7, exhibits energy primarily at low frequencies. In contrast, the acoustic response shown in FIG. 8 exhibits energy primarily at high frequencies. This response was measured while the head was exposed to an external sound field produced by a speaker driven by white noise and is indicative of its modal acoustic and blast response. Analysis of these and other results determined an appropriate cutoff frequency to separate impact and blast events would be 100 Hz.

While these results suggest the frequency response of the hearable accelerometers may be used to separate and categorize impulse and blast events, the question remains as to whether the accelerometer's pressure response is representative of the head's pressure response. The two should be well correlated if the accelerometer response is to be used as a basis for calculating blast exposure risk metrics.

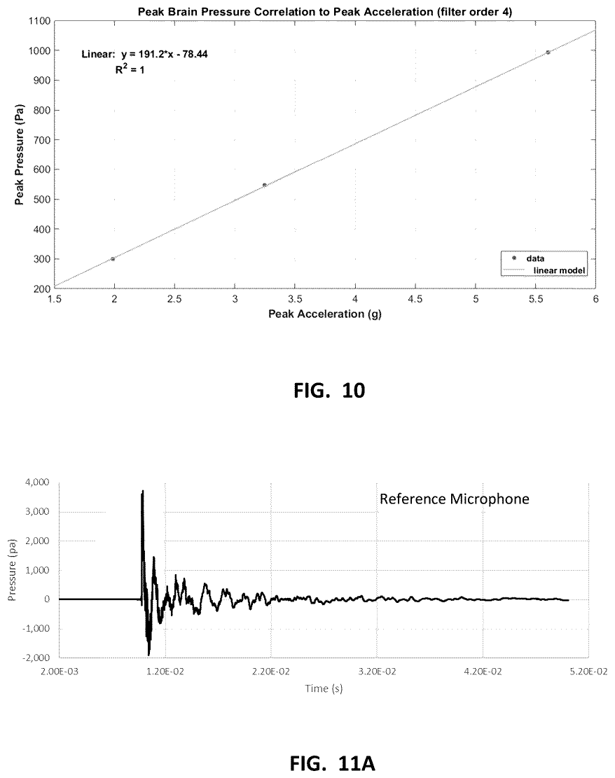

While most blast studies focus on the external blast wave outside the head, the present invention also considers the pressure wave inside the brain, which is the injury site of concern. FIG. 9 shows pressure measurements made external to the head and inside the brain of the biofidelic head simulator exposed to sound from a gunshot. The brain pressure wave is much different from the external blast, having been modified by the skull. The earplug accelerometer signal is also shown, and it exhibits a waveshape with frequency content similar to that of the brain pressure wave. FIG. 10 shows a correlation between the earplug acceleration response and the brain pressure response for gunshots of different peak overpressures. The two are well correlated, indicating that earplug acceleration may be used as a basis to calculate blast risk metrics.

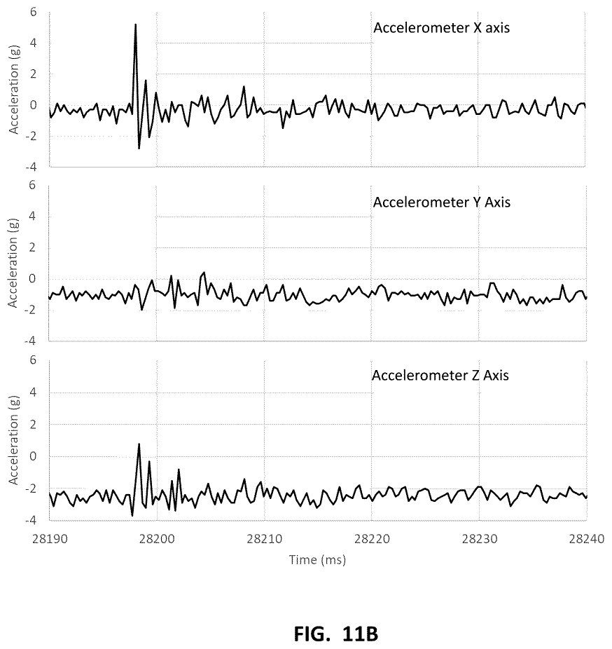

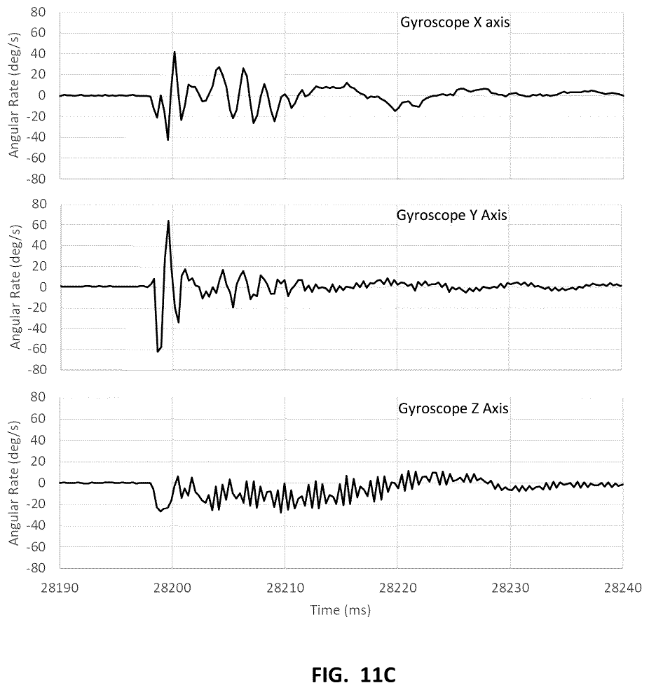

In an exemplary embodiment, the single axis, high bandwidth accelerometer is replaced by a triaxial accelerometer 121 and a triaxial gyroscope within an IM U 122. These sensors also exhibit a pressure response, as shown in FIG. 11A, FIG. 11B, and FIG. 11C. This response was recorded with the sensors fixed to a heavy base and exposed to gunshot sound from a starter's pistol. The pressure wave from the gunshot is shown in FIG. 11A. The response of the accelerometer and gyroscope sensors mounted in hearable earpieces and fitted to the ear of the biofidelic head simulator are shown in FIG. 11B and FIG. 11C, respectively. The pressure impulse is apparent in these traces. For each sensor, one axis exhibits a greater response than the others.

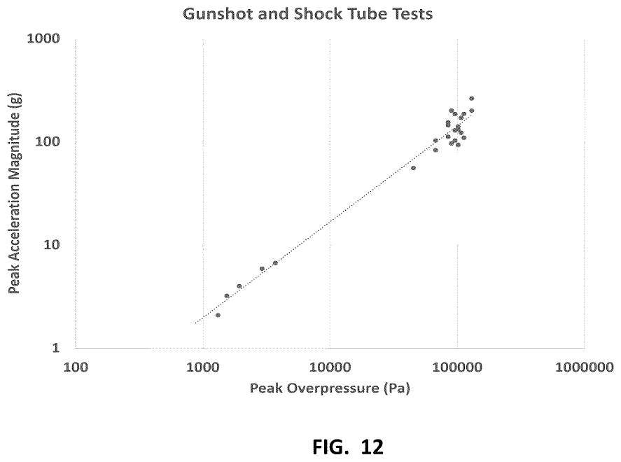

The peak sound pressure level from a starter's pistol gunshot is approximately 160 dB SPL which is at the low end of impulse sound and blast exposures. Tests in a shock tube demonstrated that the pressure sensitivity of motion sensors could be used to measure exposure to higher level blast overpressures. For these tests, the hearable earpiece was fitted to a model ear positioned at the exit of a shock tube. The results are shown in FIG. 12, where the shock tube data are plotted along with the gunshot data. The peak acceleration measured by the earpiece accelerometers shows good correlation with the peak overpressure of the blast wave. Angular rate data from the gyroscope were not as well correlated, so the accelerometer was selected as the sensor of choice in this embodiment. These results indicate that the motion sensors used for impact measurement may also be used for blast measurement. The range of hearable exposure measurements may, therefore, be extended from the 150 dB (0.36 Pa, 0.00005 psi) limit of the internal microphone up to more than 194 dB (100,000 Pa, 15 psi) depending on the pressure sensitivity of the motion sensors selected. This additional capability is gained without the need to introduce an additional sensor and data acquisition channel, thus remaining within the severe space and power constraints of a wireless hearable device.

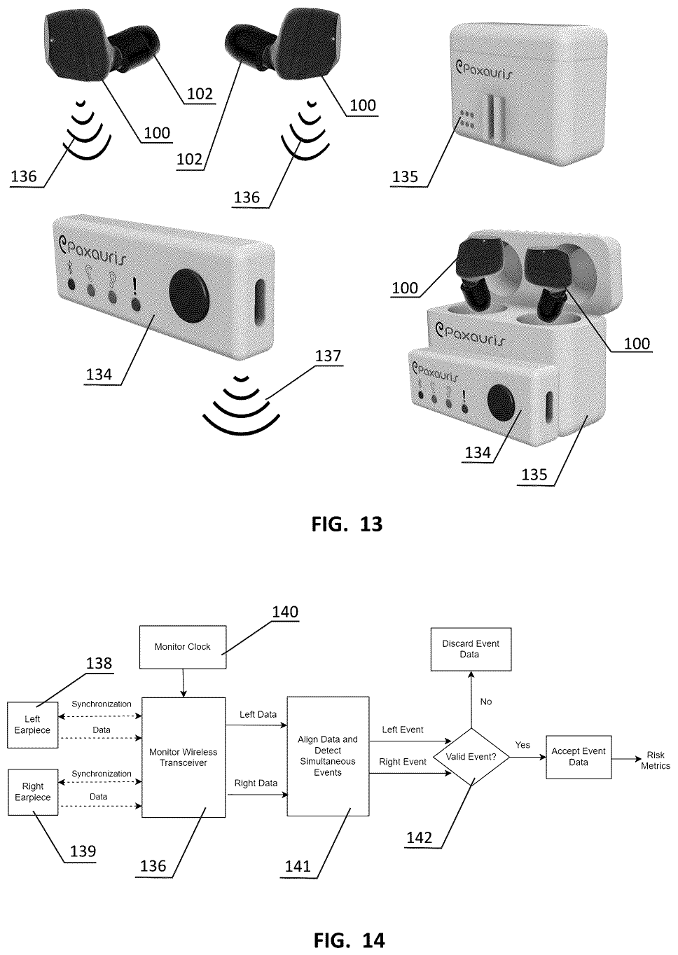

An embodiment of the complete hearable system is shown in FIG. 13. The system consists of two wireless in-ear hearables with instrumented earpieces 100 (left and right) and noise attenuating eartips 102, a head-mounted monitor 134, and a storage and charging case 135. The wireless earpieces are instrumented with sensors to measure exposure risks. They also provide level-dependent hear-through for clear communications and enhanced situational awareness. Exposure data are transmitted from the earpieces 100 to the head-mounted monitor 134 using a robust and covert near-field communications (NFC) link 136. Other wireless modalities such as Bluetooth and the like may also be employed, in which case the monitor could be any device with wireless connectivity, such as a phone, tablet, laptop, or other device. The monitor 134 processes and records the data and warns of exposure exceedances. A separate wireless link 137 may be used to upload data to a site's Environmental Health and Safety (EHS) management system. The hearable storage case 135 may be connected to the monitor 134 for USB or other charging.

The monitor 134 processes data received from both left and right hearable earpieces 100 and presents the risk metrics to the user. An aspect of the present invention is that monitor processing algorithms include checks to identify false noise, impact, or blast events. An example embodiment of such checks is shown in FIG. 14. The clocks in the left earpiece 138 and the right earpiece 139 are synchronized with the monitor clock 140 through a timing signal transmitted over the NFC wireless link 136 or another method. The exposure data transmitted to the monitor from the left and right earpieces are then aligned and simultaneous events detected 141. The event parameters are then compared to see if they agree to within a pre-determined range to see if they are valid 142. Simultaneous events with parameters that agree within this predetermined range are accepted and those with values outside the range are discarded. For example, head shadow and transcranial attenuation limit the maximum relative difference expected between left and right measurements. An example threshold might be 40 dB. In this case, if the right earpiece recorded an impulse sound event with a peak magnitude of 180 dB, and the left earpiece did not record a matching event or recorded a matching event with a peak magnitude of 120 dB, then the event would be rejected. A similar method would apply to impact event. False event detection due to bumps is an issue for body-worn dosimeters. This aspect of the invention, made possible through the redundancy of left and right hearables, overcomes this issue.

The hearing protection provided by the hearable eartips depends on the hearables being fitted correctly. Valid exposure measurements also depend on the hearables being fitted correctly. An aspect of this invention is that the difference in sound level between the internal microphone and the external microphone provides a measure of the attenuation provided by the ear tips, which is a measure of the fit. Acceptable attenuation means the eartips are well fitted and the hearable is secure and well coupled to the head, which means the head motion measurements and the impact exposure metrics are accurate. This aspect of the invention overcomes a significant issue with other head impact measurements systems, which can provide inaccurate and false metrics when they are not well coupled to the head.

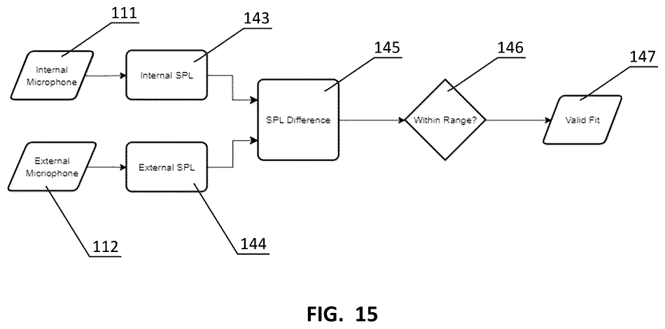

An embodiment of this hearable fit check is shown in FIG. 15. The signal from the internal microphone 111 is converted into a sound pressure level (SPL) 143. The signal from the external microphone 112 is similarly converted to SPL 144. The difference between these two SPLs is computed 145 and compared to a predetermined minimal acceptable value appropriate for the eartip 146. If the difference is greater than the minimal acceptable value a valid fit is determined, and exposure data are flagged as valid 147. In this embodiment, if the fit is not valid, an audible or other warning may be presented to the user via the earpiece speaker or via a visual or haptic warning from the monitor.

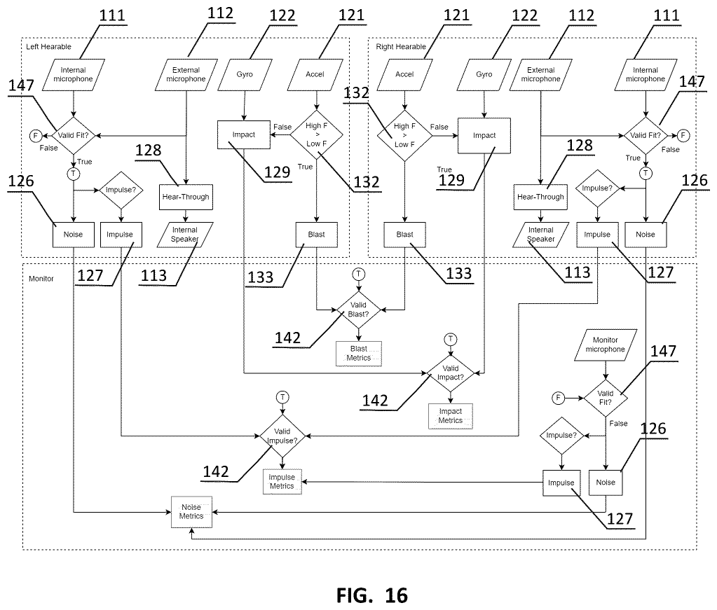

The data and logic flow in an embodiment of the hearable system is shown in FIG. 16. Signals from the internal microphones 111 and the external microphone 112 in the left and right earpieces are processed by the fit-check function 147 and if the fit is valid, the signals from the internal microphone 111 are processed by the continuous noise dosimetry function 126 and the impulse detection and measurement function 127. If the fit is not valid, the monitor microphone is used to compute the continuous and impulse noise metrics. With this approach, if the wearer's earplugs are not correctly fitted, or are removed and stored in their pocket, their actual unprotected noise exposure will be captured by the monitor microphone.

The signal from the external microphone 112 is also processed through the hear-through algorithm 128 and the safe, level-dependent signal is output to the internal speaker 113.

Signals from the accelerometers 121 are processed through the frequency-dependent classification function 132. Those with more high-frequency energy classified as blast events are processed through the blast correlation function 133 while those with more low-frequency energy are processed through the impact measurement function 129.

The impulse, impact, and blast events detected by the left and right earpieces are transmitted to the monitor where the events from each earpiece are aligned and compared in the event matching function 142, and those determined to be valid are recorded and reported by the monitor.

In various embodiments, an in-ear hearable device for monitoring exposure to continuous noise, impulse noise, blast overpressure, and head impacts comprises a left earpiece and a right earpiece each shaped to fit in the concha of the respective ear of a wearer, with the left earpiece and right earpiece each including: a sound port tube extending from an earpiece interior into an ear canal of the wearer; a sound-attenuating eartip fitted to the sound port tube and providing hearing protection without blocking the sound port tube; one or more internal acoustic sensors coupled to the sound port tube for measuring continuous and impulse noise exposure; and one or more motion sensors to detect one or more of linear accelerations and angular rates for measuring head impact exposure.

In various embodiments, at least one processor is further included, wherein the at least one processor is configured to process a function to receive an analog or digital signal from the one or more internal acoustic sensors and the one or more motion sensors, and to compute exposure metrics for continuous noise, impulse noise, blast overpressure, and head impacts.

In various embodiments, the at least one processor is further configured to process at least one filter applied to the signal from the one or more motion sensors to determine low frequency energy in the signal; at least one filter applied to the signal from the one or more motion sensors to determine high frequency energy in the signal; and a function to detect and classify events based on level and frequency content of filtered motion sensor signals, wherein the filtered motion sensor signals with greater energy at low frequencies are classified as impact events and the filtered motion signals with greater energy at high frequencies are classified as blast events.

In various embodiments, the at least one processor is further configured to process one or more functions to compute head impact exposure metrics from the filtered motion sensor signals.

In various embodiments, the at least one processor is further configured to process one or more functions to compute blast exposure metrics from the filtered motion sensor signals.

In various embodiments, the computation of blast exposure metrics is based on correlations between motion sensor data and blast overpressure data with the left earpiece and the right earpiece fitted into respective ears of instrumented biofidelic head fixtures or an instrumented post-mortem-human-subject head.

In various embodiments, one or more external acoustic sensors are provided in each of the left and right earpieces, coupled through an external port to an environment outside the ear of the wearer.

In various embodiments, at least one processor is configured to process a function to determine a difference between a sound level computed from an internal microphone and an external microphone in each of the left and right earpieces and compare the difference to a predetermined attenuation threshold to determine whether one or both of the left earpiece and the right earpiece is properly fitted.

In various embodiments, only motion sensor data from properly fitted earpieces are included in head impact and blast exposure metrics.

In various embodiments, only acoustic sensor data from properly fitted earpieces are included in continuous noise and impulse noise exposure metrics.

In various embodiments, one or more speakers are provided in each of the left and right earpieces acoustically coupled to the sound port tube.

In various embodiments, at least one processor is configured to process one or more functions to receive a signal from the external acoustic sensor in each of the left and right earpieces and apply a gain that is dependent on a level of the signal before outputting the signal to the one or more speakers such that the level is limited to a predetermined safe level.

In various embodiments, the one or more speakers provide an aural warning to alert the wearer of exceedances for continuous noise, impulse noise, head impact, or blast exposures.

In various embodiments, at least one processor is configured to process one or more functions to detect and compare simultaneous impulse, impact, and blast events from either the left earpiece or the right earpiece and to reject events that do not have a matching event detected from another of the left or right earpiece within a predetermined magnitude range.

In various embodiments, the predetermined magnitude range is determined from a transcranial attenuation and a head shadowing effect corresponding to a type of event detected with different ranges for impulse, impact, and blast events.

In various embodiments, an in-ear hearable device for monitoring exposure to continuous noise, impulse noise, blast overpressure, and head impacts comprises a left earpiece and a right earpiece each shaped to fit in a concha of a respective ear of a wearer, with the left earpiece and right earpiece each including a sound port tube extending from an earpiece interior into an ear canal of the wearer; a sound-attenuating eartip fitted to the sound port tube and providing hearing protection without blocking the sound port tube; one or more microphones coupled to the sound port tube for measuring continuous and impulse noise exposure; and one or more accelerometers or gyroscopes to detect one or more of linear accelerations and angular rates for measuring head impact exposure. The in-ear hearable device further comprises at least one processor, wherein the at least one processor is configured to process a function to receive analog signals or digital signals from one or more internal acoustic sensors and one or more motion sensors, and compute exposure metrics for continuous noise, impulse noise, blast overpressure, and head impacts.

In various embodiments, the at least one processor is further configured to process at least one filter applied to the analog or digital signals from the one or more motion sensors to determine low frequency energy; at least one filter applied to the analog or digital signals from the one or more motion sensors to determine high frequency energy; and a function to detect and classify events based on level and frequency content of filtered motion sensor signals, wherein filtered motion sensor signals with greater energy at low frequencies are classified as impact events and filtered motion sensor signals with greater energy at high frequencies are classified as blast events.

In various embodiments, the at least one processor is further configured to process one or more functions to compute head impact exposure metrics from the filtered motion sensor signals.

In various embodiments, the at least one processor is further configured to process one or more functions to compute blast exposure metrics from the filtered motion sensor signals.

In various embodiments, the computation of the blast exposure metrics is based on correlations between motion sensor data and blast overpressure data with the left earpiece and the right earpiece fitted into respective ears of instrumented biofidelic head fixtures or an instrumented post-mortem-human-subject head.

In various embodiments, one or more external acoustic sensors are provided in each of the left and right earpieces, coupled through an external port to an environment outside the ear of the wearer.

In various embodiments, the at least one processor in each of the left and right earpieces is configured to process a function to determine a difference between a sound level computed from the one or more microphones and an external microphone and compare the difference to a predetermined attenuation threshold to determine whether one or both of the left earpiece and the right earpiece is properly fitted.

In various embodiments, only accelerometer and gyroscope data from properly fitted earpieces are included in head impact and blast exposure metrics.

In various embodiments, only microphone data from properly fitted earpieces are included in continuous noise and impulse noise exposure metrics.

In various embodiments, one or more speakers are provided in each of the left and right earpieces acoustically coupled to the sound port tube.

In various embodiments, the at least one processor in each of the left and right earpieces is configured to process a function to receive a signal from the external microphone and apply a gain that is dependent on a level of the signal before outputting the signal to the speaker such that the level is limited to a predetermined safe level.

In various embodiments, the one or more speakers provide an aural warning to alert the wearer of exceedances for continuous noise, impulse noise, head impact, or blast exposures.

In various embodiments, a wireless transceiver is provided and configured to communicate exposure metrics to a head or body-mounted monitor.

In various embodiments, the wireless communication method is a near-field communication method for establishing a body-area network with a range of less than approximately 30 inches.

In various embodiments, the wireless transceiver is configured to send and receive synchronization signals between each left and right earpiece and the head or body-mounted monitor.

In various embodiments, a wired communication method is employed rather than wireless communication.

In various embodiments, the head or body-mounted monitor is configured to provide one or more of visual, tactile, and audible warnings of exposure exceedances.

In various embodiments, at least one processor is provided in the head or body-mounted monitor to process one or more functions to detect and compare simultaneous impulse, impact, and blast events from either the left earpiece or the right earpiece, and to reject events that do not have a matching event detected from another of the left and right earpieces within a predetermined magnitude range.

In various embodiments, the predetermined magnitude range is determined from a transcranial attenuation and a head shadowing effect corresponding to a type of event detected with different ranges for impulse, impact, and blast events.

Various devices, systems, and components can be included and adapted to process and carry out the aspects, computations, functions, and algorithmic processing of the software systems and methods of the present invention. Computing systems and devices of the present invention may include a computing processor, which may include one or more microprocessors and/or one or more circuits, such as an application specific integrated circuit (ASIC), field-programmable gate arrays (FPGAs), etc. Further, the devices can include a network interface. The network interface is configured to enable communication with a communication network, other devices, components, and systems, using a wired and/or wireless connection.

The devices or systems of the present invention can include memory, such as non-transitive, which may include one or more non-volatile storage devices and/or one or more volatile storage devices (e.g., random access memory (RAM)). In instances where the devices include a computing processor, computer readable program code may be stored in a computer readable medium or memory. The computer program or software code can be stored on a tangible, or non-transitive, machine-readable medium or memory. In some embodiments, computer readable program code is configured such that when executed by a processor, the code causes the device or system to perform the steps, processing, and functions described above and herein. In other embodiments, the device or system is configured to perform steps described herein without the need for code.

It will be recognized by one skilled in the art that operations, functions, algorithms, logic, method steps, routines, sub-routines, and modules may be implemented in software, in firmware, in special purpose digital logic, and any combination thereof without deviating from the spirit and scope of the present invention as recited within the claims attached hereto.

The present invention may be embodied in other specific forms without departing from the spirit or essential attributes thereof, and it is, therefore, desired that the present embodiment be considered in all respects as illustrative and not restrictive. Similarly, the above-described methods and techniques for providing and using the present invention are illustrative processes and are not intended to limit the methods of manufacturing the present invention to those specifically defined herein. Further, features and aspects, in whole or in part, of the various embodiments described herein can be combined to form additional embodiments within the scope of the invention even if such combination is not specifically described herein.

For purposes of interpreting the claims for the present invention, it is expressly intended that the provisions of Section 112(f) of 35 U.S.C. are not to be invoked unless the specific terms “means for” or “step for” are recited in a claim.

What is claimed is:

1. An in-ear hearable device for monitoring exposure to continuous noise, impulse noise, blast overpressure, and head impacts, comprising:
a left earpiece and a right earpiece each shaped to fit in a concha of a respective ear of a wearer, with the left earpiece and right earpiece each including:
a sound port tube extending from an earpiece interior into an ear canal of the wearer;
a sound-attenuating eartip fitted to the sound port tube and providing hearing protection without blocking the sound port tube;
one or more internal acoustic sensors coupled to the sound port tube for measuring continuous and impulse noise exposure; and
one or more motion sensors to detect one or more of linear accelerations and angular rates for measuring head impact exposure.

2. The device of claim 1, further including at least one processor, wherein the at least one processor is configured to process a function to receive an analog or digital signal from the one or more internal acoustic sensors and the one or more motion sensors, and to compute exposure metrics for continuous noise, impulse noise, blast overpressure, and head impacts.

3. The device of claim 2, wherein the at least one processor is further configured to process:
at least one filter applied to the signal from the one or more motion sensors to determine low frequency energy in the signal;
at least one filter applied to the signal from the one or more motion sensors to determine high frequency energy in the signal; and
a function to detect and classify events based on level and frequency content of filtered motion sensor signals, wherein the filtered motion sensor signals with greater energy at low frequencies are classified as impact events and the filtered motion signals with greater energy at high frequencies are classified as blast events.

4. The device of claim 3, wherein the at least one processor is further configured to process one or more functions to compute head impact exposure metrics from the filtered motion sensor signals.

5. The device of claim 3, wherein the at least one processor is further configured to process one or more functions to compute blast exposure metrics from the filtered motion sensor signals.

6. The device of claim 5, wherein the computation of blast exposure metrics is based on correlations between motion sensor data and blast overpressure data with the left earpiece and the right earpiece fitted into respective ears of instrumented biofidelic head fixtures or an instrumented post-mortem-human-subject head.

7. The device of claim 1, further comprising one or more external acoustic sensors in each of the left and right earpieces, coupled through an external port to an environment outside the ear of the wearer.

8. The device of claim 7, further comprising at least one processor configured to process a function to determine a difference between a sound level computed from an internal microphone and an external microphone in each of the left and right earpieces and compare the difference to a predetermined attenuation threshold to determine whether one or both of the left earpiece and the right earpiece is properly fitted.

9. The device of claim 8, wherein only motion sensor data from properly fitted earpieces are included in head impact and blast exposure metrics.

10. The device of claim 8, wherein only acoustic sensor data from properly fitted earpieces are included in continuous noise and impulse noise exposure metrics.

11. The device of claim 7, further comprising one or more speakers in each of the left and right earpieces acoustically coupled to the sound port tube.

12. The device of claim 11, further including at least one processor, wherein the at least one processor is configured to process one or more functions to receive a signal from the external acoustic sensor in each of the left and right earpieces and apply a gain that is dependent on a level of the signal before outputting the signal to the one or more speakers such that the level is limited to a predetermined safe level.

13. The device of claim 11, wherein the one or more speakers provide an aural warning to alert the wearer of exceedances for continuous noise, impulse noise, head impact, or blast exposures.

14. The device of claim 1, further comprising at least one processor configured to process one or more functions to detect and compare simultaneous impulse, impact, and blast events from either the left earpiece or the right earpiece and to reject events that do not have a matching event detected from another of the left or right earpiece within a predetermined magnitude range.

15. The device of claim 14, wherein the predetermined magnitude range is determined from a transcranial attenuation and a head shadowing effect corresponding to a type of event detected with different ranges for impulse, impact, and blast events.

16. An in-ear hearable device for monitoring exposure to continuous noise, impulse noise, blast overpressure, and head impacts, comprising:
a left earpiece and a right earpiece each shaped to fit in a concha of a respective ear of a wearer, with the left earpiece and right earpiece each including:
a sound port tube extending from an earpiece interior into an ear canal of the wearer;
a sound-attenuating eartip fitted to the sound port tube and providing hearing protection without blocking the sound port tube;
one or more microphones coupled to the sound port tube for measuring continuous and impulse noise exposure; and
one or more accelerometers or gyroscopes to detect one or more of linear accelerations and angular rates for measuring head impact exposure;
at least one processor, wherein the at least one processor is configured to process a function to receive analog signals or digital signals from one or more internal acoustic sensors and one or more motion sensors, and compute exposure metrics for continuous noise, impulse noise, blast overpressure, and head impacts.

17. The device of claim 16, wherein the at least one processor is further configured to process:
at least one filter applied to the analog or digital signals from the one or more motion sensors to determine low frequency energy;
at least one filter applied to the analog or digital signals from the one or more motion sensors to determine high frequency energy; and
a function to detect and classify events based on level and frequency content of filtered motion sensor signals, wherein filtered motion sensor signals with greater energy at low frequencies are classified as impact events and filtered motion sensor signals with greater energy at high frequencies are classified as blast events.

18. The device of claim 17, wherein the at least one processor is further configured to process one or more functions to compute head impact exposure metrics from the filtered motion sensor signals.

19. The device of claim 17, wherein the at least one processor is further configured to process one or more functions to compute blast exposure metrics from the filtered motion sensor signals.

20. The device of claim 19, wherein the computation of the blast exposure metrics is based on correlations between motion sensor data and blast overpressure data with the left earpiece and the right earpiece fitted into respective ears of instrumented biofidelic head fixtures or an instrumented post-mortem-human-subject head.

21. The device of claim 16, further comprising one or more external acoustic sensors in each of the left and right earpieces, coupled through an external port to an environment outside the ear of the wearer.

22. The device of claim 21, wherein the at least one processor in each of the left and right earpieces is configured to process a function to determine a difference between a sound level computed from the one or more microphones and an external microphone and compare the difference to a predetermined attenuation threshold to determine whether one or both of the left earpiece and the right earpiece is properly fitted.

23. The device of claim 22, wherein only accelerometer and gyroscope data from properly fitted earpieces are included in head impact and blast exposure metrics.

24. The device of claim 22, wherein only microphone data from properly fitted earpieces are included in continuous noise and impulse noise exposure metrics.

25. The device of claim 21, further comprising one or more speakers in each of the left and right earpieces acoustically coupled to the sound port tube.

26. The device of claim 25, wherein the at least one processor in each of the left and right earpieces is configured to process a function to receive a signal from the external microphone and apply a gain that is dependent on a level of the signal before outputting the signal to the speaker such that the level is limited to a predetermined safe level.

27. The device of claim 25, wherein the one or more speakers provide an aural warning to alert the wearer of exceedances for continuous noise, impulse noise, head impact, or blast exposures.

28. The device of claim 16, further comprising a wireless transceiver configured to communicate exposure metrics to a head or body-mounted monitor.

29. The device of claim 28, wherein a wireless communication method is a near-field communication method for establishing a body-area network with a range of less than approximately 30 inches.

30. The device of claim 28, wherein the head or body-mounted monitor is configured to provide one or more of visual, tactile, and audible warnings of exposure exceedances.

31. The device of claim 28, wherein the wireless transceiver is configured to send and receive synchronization signals between each left and right earpiece and the head or body-mounted monitor.

32. The device of claim 31, further comprising at least one processor in the head or body-mounted monitor to process one or more functions to detect and compare simultaneous impulse, impact, and blast events from either the left earpiece or the right earpiece, and to reject events that do not have a matching event detected from another of the left and right earpieces within a predetermined magnitude range.

33. The device of claim 32, wherein the predetermined magnitude range is determined from a transcranial attenuation and a head shadowing effect corresponding to a type of event detected with different ranges for impulse, impact, and blast events.
